(19) United States
(12) Patent Application Publication
(10) Pub. No.: US 2012/0190719 A1
(43) Pub. Date: Jul. 26, 2012

(54) METHODS OF TREATING MOTOR DISORDERS WITH ALPHA-2B ANDRENERGIC RECEPTOR AGONISTS

(75) Inventors: Daniel W. Gil, Corona Del Mar, CA (US); John E. Donello, Dana Point, CA (US); Lauren M.B. Luhrs, Rancho Santa Margarita, CA (US)

(73) Assignee: ALLERGAN, INC., Irvine, CA (US)

(21) Appl. No.: 13/389,338

(22) PCT Filed: Aug. 26, 2010
(86) PCT No.: PCT/US2010/046860

§ 371 Date: Mar. 14, 2012

Related U.S. Application Data
(60) Provisional application No. 61/237,165, filed on Aug. 26, 2009.

(51) Int. Cl. A61K 31/4174; A61K 31/4164; A61P 25/18; A61P 25/00; A61P 25/30; A61K 31/17; A61K 31/4168

(52) U.S. Cl. 514/392; 514/587; 514/396

(57) ABSTRACT

Disclosed herein is a method of treating compulsive disorders comprising administering to a subject in need of such treatment an alpha-2 receptor agonist.

### RELATED APPLICATION DATA

This application claims the benefit of U.S. Provisional patent application Ser. No. 61/237,165, filed Aug. 26, 2009, which is hereby incorporated by reference in its entirety.

### CROSS-REFERENCE

Disclosed herein is a method of treating motor disorders by administering to a subject an alpha-2 adrenergic receptor agonist. Such agonists are effective in treating the disorders without sedating the patient to whom they are administered.

### DETAILED DESCRIPTION OF THE INVENTION

### Alpha-2 Receptor Agonists

Alpha-2 receptor agonists are those compounds that activate alpha-2 adrenergic receptors. There are three subtypes of this receptor, designated A, B, and C. A compound is an “alpha-2B receptor agonist” if it has greater than 25% efficacy relative to brimonidine at the alpha-2B adrenergic receptor; a compound is an “alpha-2C receptor agonist” if it has greater than 25% efficacy relative to brimonidine at the alpha-2C adrenergic receptor; and a compound is an “alpha-2B/2C receptor agonist” if it has greater than 25% efficacy relative to brimonidine at both the alpha-2B and alpha-2C adrenergic receptors.

In one embodiment, the methods of the present invention use alpha-2 agonists lacking significant activity at the alpha-2A receptor subtype. An agonist lacks significant alpha-2A receptor activity if the agonist has less than 40% of the efficacy of brimonidine at the alpha-2A receptor subtype. Compounds of the invention include, therefore, alpha-2B receptor agonists lacking significant alpha-2A activity; alpha 2B/2C receptor agonists lacking significant alpha-2A activity; and alpha-2C receptor agonists lacking significant alpha-2A activity. Any of the foregoing compounds may be used, even if they bind receptors other than alpha-2 receptors; for example, alpha-1 receptor agonists may be used, provided that the alpha-1 agonists also have greater than 25% efficacy relative to brimonidine at one or both of the alpha-2B and alpha-2C receptor subtypes, and lack significant alpha-2A receptor activity.

Efficacy, also known as intrinsic activity, is a measure of maximal receptor activation achieved by a compound and can be determined using any accepted assay of alpha-adrenergic receptor activation, such as a cAMP or Receptor Selection and Amplification Technology (RSAT). Efficacy is represented as a ratio or percentage of the maximal effect of the drug to the maximal effect of a standard agonist for each receptor subtype. Brimonidine, itself an alpha-2B receptor agonist (it is has 100% the efficacy of brimonidine at the alpha-2B adrenergic receptor), is used as the standard agonist for the alpha-2B adrenergic receptors.

Agonist activity can be characterized using any of a variety of routine assays, including, for example, Receptor Selection and Amplification Technology (RSAT) assays (Messier et al., Pharmacol. Toxicol. 76:308-11 (1995); cyclic AMP assays (Shimizu et al., J. Neurochem. 16:1609-1619 (1969)); and cytosensor microphysiometry assays (Neve et al., J. Biol. Chem. 267:25748-25753 (1992)). Such assays generally are performed using cells that naturally express only a single alpha-adrenergic receptor subtype, or using transfected cells expressing a single recombinant alpha-adrenergic receptor subtype. The adrenergic receptor can be a human receptor or homolog of a human receptor having a similar pharmacology.

The RSAT assay measures receptor-mediated loss of contact inhibition resulting in selective proliferation of receptor-containing cells in a mixed population of confluent cells. The increase in cell number is assessed with an appropriate detectable marker gene such as beta-galactosidase, if desired, in a high throughput or ultra high throughput assay format. Receptors that activate the G protein, Gq, elicit the proliferative response. Alpha-adrenergic receptors, which normally couple to Gi, activate the RSAT response when coexpressed with a hybrid Gq protein containing a Gi receptor recognition domain, designated Gq/i5. Conklin et al., Nature 363:274-6 (1993)).

As an example, an RSAT assay can be performed essentially as follows. NIH-3T3 cells are plated at a density of 2×10⁶ cells in 15 cm dishes and maintained in Dulbecco's modified Eagle's medium supplemented with 10% calf serum. One day later, cells are cotransfected by calcium phosphate precipitation with mammalian expression plasmids encoding p-SV-β-galactosidase (5-10 μg), receptor (1-2 μg) and G protein (1-2 μg). Carrier DNA, for example 40 μg salmon sperm DNA, also can be included to increase transfection efficiency. Fresh media is added on the following day; one to two days later, cells are harvested and frozen in 50 assay aliquots. Transfected cells are thawed, and 100 μl of cells added to 100 μl aliquots of compound to be tested, with various concentrations assayed in triplicate, for example, in 96-well plates. Incubation continues for 72 to 96 hours at 37° C. After washing with phosphate-buffered saline, β-galactosidase activity is determined by adding 200 μl of chromogenic substrate (3.5 mM O-nitrophenyl-β-D-galactopyranoside/0.5% NP-40 in phosphate buffered saline), incubating overnight at 30° C., and measuring optical density at 420 nm. The absorbency is a measure of enzyme activity, which depends on cell number and reflects receptor-mediated cell proliferation. The EC₅₀ and maximal effect (i.e., efficacy) of each drug at each receptor is determined.

Alpha-2B and -2C receptor agonists, including those lacking significant alpha-2A receptor activity, are known in the art. Detailed information regarding alpha-2 agonists, including their structure, synthesis, and activity, may be found in U.S. Pat. No. 6,329,369, No. 6,534,542, No. 6,545,182, No. 6,787,517, No. 6,841,684, and No. 7,091,232; in U.S. Patent Application Publication No. 2003/0092766, No. 2004/0132824, No. 2004/0220402, No. 2005/0075366, and No. 2005/0267186; and in U.S. patent application Ser. No. 11/172,229, Ser. No. 11/232,323, Ser. No. 11/232,341, No. 60/613,870, No. 60/695,650, No. 60/747,444, No. 60/884,718, No. 60/917,828, No. 60/911,422, No. 60/911,478, and No. 60/948,389, the disclosures of all which are incorporated herein by reference.

One can use in the methods of the invention any pharmaceutically acceptable salt, prodrug, isomer, or racemate of any alpha-2 receptor agonist.

### Pharmaceutically Acceptable Salts

Alpha-2 receptor agonists may be used as their pharmaceutically acceptable salts.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the compositions and methods of the invention a prodrug of any alpha-2 receptor agonist.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

The alpha-2 receptor agonists of the invention may be either synthetically produced, or may be produced within the body after administration of a prodrug. Hence, the term “alpha-2 receptor agonist” encompasses both compounds produced by a manufacturing process and those compounds formed in vivo only when another drug administered.

### Isomers and Racemates

One can use in the compositions and methods of the invention an enantiomer, stereoisomer, or other isomer of an alpha-2 receptor agonist. One can also use in the compositions and methods of the invention a racemic mixture or one or both racemates, in any proportion.

### Dose

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art. In general, alpha-2 receptor agonists are administered in therapeutically effective doses, that is, at a dose that is sufficient to produce the desired therapeutic effect.

### Excipients and Dosage Forms

Those skilled in the art will readily understand that alpha-2 receptor agonists can be admixed with pharmaceutically acceptable excipients which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, No. 4,166,452, and No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

### Compulsive Disorders

Compounds of the invention are useful in treating compulsive disorders. To “treat,” as used here, means to deal with medically. It includes administering an alpha-2B receptor agonist to prevent the onset of a condition, to diminish its severity, and to prevent its reoccurrence. The inventors have discovered that the compounds of the invention may be used to treat compulsive disorders without causing the sedation that ordinarily accompanies the administration of alpha-2 agonists.

A “compulsive disorder,” as that term is used here, is any condition characterized by irresistible impulsive behavior. Examples of compulsive disorders include, but are not limited to obsessive compulsive disorder, eating disorders (including but not limited to anorexia nervosa, bulimia nervosa), pathological gambling, body dysmorphic disorder (including but not limited to olfactory reference syndrome and muscle dysmorphia), trichotillomania, dermatillomania, compulsive buying disorder, kleptomania, hypochondriasis, depersonalization disorder, Sydenham's chorea, torticollis, sexual addiction, drug addiction, and self injurious behaviours.

### EXAMPLES

The invention is illustrated by the following examples. This is provided for illustration only; many more embodiments are possible.

The usefulness of alpha-2B receptor agonists in treating compulsive disorders are exemplified in a widely-used rodent model of compulsive behavior, the marble burying task. Absence of alpha2B receptor activity, either following treatment with an alpha2B receptor antagonist or in alpha2B receptor knockout mice, results in increased compulsive behavior in the marble burying task. Hence, alpha2B receptor activation with an alpha2B receptor agonist will reduce compulsive behavior. Two alpha2B agonists used by the inventors, each belonging to a different compound class, are: Compound A, an imidazole thione, and Compound B, an imidazole amine.

Importantly, these compounds are orally active, and therefore could be administered in solution, tablet or capsule. These compounds are not sedating at therapeutic doses following oral administration.

Table 1, below, shows the structures of Compounds A and B:

| COMPOUND | COMPOUND STRUCTURE |
|---|---|
| A | 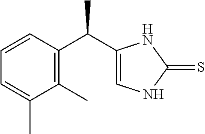 |
| B | 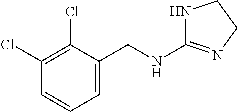 |

### General Findings

TABLE 2
α_{2B}-KO-selective mice exhibit compulsive burying behavior
Mice were exposed to the defensive burying task (described
below). Alpha 2B KO mice exhibited increased burying
behavior relative to their WT counterparts. [P = 0.049
Unpaired T-test, 2B WT vs 2B KO]. N = 11-12/group.

| | % | STANDARD |
|---|---|---|
| | BURIED | ERROR |
| WT | 51.8% | 3.6% |
| 2B WT | 47.6% | 6.4% |
| 2A KO | 33.9% | 3.8% |
| 2B KO | 63.6%* | 4.0% |
| 2C KO | 49.4% | 7.2% |

TABLE 3
α_{2B}-antogonist-treated mice exhibit compulsive burying
behavior

| | | Standard |
|---|---|---|
| | % Buried | Error |
| WT Vehicle | 46.40% | 3.10% |
| WT Compound C | 61.90%** | 3.50% |

Mice were treated with vehicle (20% DMSO in water) or Compound C, an alpha 2-B antagonist (300 ug/kg) IP 30 minutes prior to defensive burying testing:
Compound C 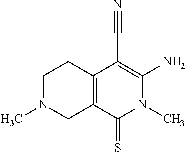
Animals treated with Compound C exhibited significantly increased burying behavior relative to vehicle-treated mice (P = 0.0078). N = 6/group

### Methods:

Defensive burying: C57B/6 male mice (25-30 g) individually placed into plexiglass mouse cages filled with standard mouse bedding in their housing room. Each cage contained 7 small marbles (diameter 1 cm) and 7 larger marbles (diameter 1.5 cm). Mice remained in the cages for 30 minutes, after which time they were removed from their home cage and the number of marbles buried more than ⅔ was counted. Animals in treatment groups (Table 3) were administered compound IP (1 ml/kg) 30 min prior to testing.

For Table 2, data were evaluated by One-way ANOVA for overall effect of genotype [F(4,57)=4.155; P=0.0053]. Because the 2B WT is the only control for the 2B KO, an unpaired T-test was used to compare differences between 2B WT and 2B KO. For Table 3, data were evaluated by unpaired T-Test.

In sum, the method of the invention comprises the following:

1. A method of treating a compulsive disorder, the method comprising administering to a subject in need of such treatment an alpha-2 receptor agonist.
2. The method of 1, wherein the alpha-2 receptor agonist lacks significant alpha-2A receptor activity.
3. The method of 1 or 2, wherein the method further comprises treating the compulsive disorder without causing sedation.
4. The method of any of 1, 2, or 3, wherein the alpha-2 receptor agonist is selected from the group consisting of an alpha-2B receptor agonist and an alpha-2B/2C receptor agonist.
5. The method of any of 1, 2, or 3, wherein the alpha-2 receptor agonist is an alpha-2C receptor agonist.
6. The method of any of 1-5, wherein the disorder is selected from the group consisting of obsessive compulsive disorder, eating disorders (including but not limited to anorexia nervosa, bulimia nervosa), pathological gambling, body dysmorphic disorder (including but not limited to olfactory reference syndrome and muscle dysmorphia), trichotillomania, dermatillomania, compulsive buying disorder, kleptomania, hypochondriasis, depersonalization disorder, Sydenham's chorea, torticollis, sexual addiction, drug addiction, and self injurious behaviours.
7. The method of any of 1-6, wherein the alpha-2B receptor agonist is a compound having the following structure:

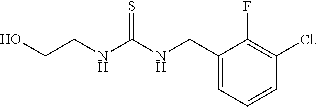

8. The method of any of 1-6, wherein the alpha-2B receptor agonist is a compound having the following structure:

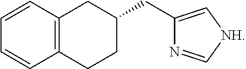

9. The method of any of 1-6, wherein the alpha-2B receptor agonist is a compound having the following structure:

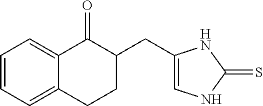

10. The method of any of 1-6, wherein the alpha-2B receptor agonist is a compound having the following structure:

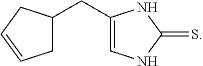

11. The method of any of 1-6, wherein the alpha-2B receptor agonist is a compound having the following structure:

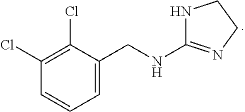

12. The method of any of 1-6, wherein the alpha-2B receptor agonist is a compound having the following structure:

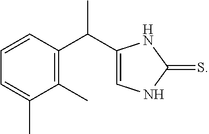

13. The method of 12, wherein the alpha-2B receptor agonist is a compound having the following structure:

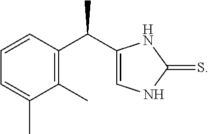

1. A method of treating a compulsive disorder, the method comprising administering to a subject in need of such treatment an alpha-2 receptor agonist.

2. The method of claim 1, wherein the alpha-2 receptor agonist lacks significant alpha-2A receptor activity.

3. The method of claim 1, wherein the method further comprises treating the compulsive disorder without causing sedation.

4. The method of claim 2, wherein the method further comprises treating the compulsive disorder without causing sedation.

5. The method of claim 4, wherein the alpha-2 receptor agonist is selected from the group consisting of an alpha-2B receptor agonist and an alpha-2B/2C receptor agonist.

6. The method of claim 4, wherein the alpha-2 receptor agonist is an alpha-2C receptor agonist.

7. The method of claim 1, wherein the disorder is selected from the group consisting of obsessive compulsive disorder, eating disorders, pathological gambling, body dysmorphic disorder, trichotillomania, dermatillomania, compulsive buying disorder, kleptomania, hypochondriasis, depersonalization disorder, Sydenham's chorea, torticollis, sexual addiction, drug addiction, and self injurious behaviours.

8. The method of claim 7, wherein the alpha-2B receptor agonist is a compound having the following structure: 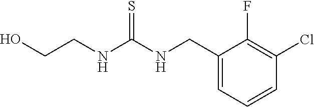
or a pharmaceutically acceptable salt thereof.

9. The method of claim 7, wherein the alpha-2B receptor agonist is a compound having the following structure: 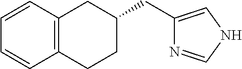
or a pharmaceutically acceptable salt thereof.

10. The method of claim 7, wherein the alpha-2B receptor agonist is a compound having the following structure: 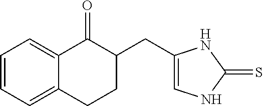
or a pharmaceutically acceptable salt thereof.

11. The method of claim 7, wherein the alpha-2B receptor agonist is a compound having the following structure: 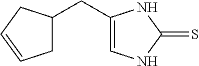
or a pharmaceutically acceptable salt thereof.

12. The method of claim 7, wherein the alpha-2B receptor agonist is a compound having the following structure: 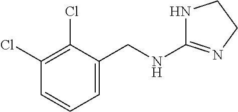
or a pharmaceutically acceptable salt thereof.

13. The method of claim 7, wherein the alpha-2B receptor agonist is a compound having the following structure: 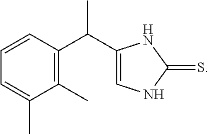
or a pharmaceutically acceptable salt thereof.

14. The method of claim 7, wherein the alpha-2B receptor agonist is a compound having the following structure: 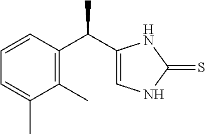
or a pharmaceutically acceptable salt thereof.
